(19) United States
(12) Patent Application Publication
(10) Pub. No.: US 2009/0093011 A1
(43) Pub. Date: Apr. 9, 2009

(54) Biosensors for ligand-directed functional selectivity

(76) Inventors: Ye Fang, Painted Post, NY (US); Ann MeeJin Ferrie, Painted Post, NY (US)

Correspondence Address: CORNING INCORPORATED, SP-TI-3-1, CORNING, NY, 14831, US

(21) Appl. No.: 11/998,521

(22) Filed: Nov. 30, 2007

Related U.S. Application Data
(60) Provisional application No. 60/997,847, filed on Oct. 5, 2007.

(51) Int. Cl. C12Q 1/02

(52) U.S. Cl. 435/29

(57) ABSTRACT

A system and method for determining ligand-directed functional selectivity of a receptor in a live-cell with a biosensor are disclosed. Also disclosed is a system and method for fragment-based screening with a cell-based, label-free biosensor functional assay.

### CLAIMING BENEFIT OF PRIOR FILED U.S. APPLICATION

This application claims the benefit of U.S. Provisional Application Ser. No. 60/997,847, filed Oct. 5, 2007. The content of this prior filed U.S. application and the entire disclosure of any publications, patents, and patent documents mentioned herein are incorporated by reference.

### BACKGROUND

The disclosure relates to the field of biosensors, specifically resonant waveguide grating (RWG) biosensors for label-free or label independent detection (LID). More particularly the disclosure relates to a system and method for determining ligand-directed functional selectivity of a receptor, for example, G protein coupled receptors (GPCRs).

### SUMMARY

The disclosure provides a system and method for determining ligand-directed functional selectivity of a receptor, for example, a G protein coupled receptors (GPCR). The disclosed method and system use molecular fragments of a natural ligand or a known ligand as a basis to determine the functional selectivity of a ligand or an array of ligands for a given GPCR, or like complex receptors. Multi-parameter analysis of dynamic mass redistribution (DMR) signals induced by, for example, a ligand individually, co-stimulated with the fragment compound of the natural ligand and a compound, or sequentially stimulated with the ligand fragment and then a compound, can provide a structure-activity relational map of the functional selectivity of the ligand and can provide design rules or guidance for, for example, new therapeutic or diagnostic agents or new uses of existing agent. The disclosure also describes a method for drug fragment screening using biosensor-based functional cell assays.

### BRIEF DESCRIPTION OF THE DRAWINGS

FIG. 1 shows exemplary β₂AR ligands and their DMR signals in quiescent A431 cells, in embodiments of the disclosure.

FIG. 2 shows a dose-dependent DMR response induced by salmeterol, as plotted as the amplitude of the P-DMR event as a function of salmeterol concentration in A431 cells, in embodiments of the disclosure.

FIG. 3 show exemplary norepinephrine-induced DMR signals, in embodiments of the disclosure.

FIG. 4 shows a relationship between the logs of apparent EC₅₀ and logs of binding affinity of selected β₂AR agonists, in embodiments of the disclosure.

FIG. 5 shows DMR signals of quiescent A431 cells induced by 2 nM epinephrine, in embodiments of the disclosure.

FIG. 6a shows a hypothetical situation where two ligands or ligand fragments (members A and B) can bind to and recognize different binding sites of a receptor, in embodiments of the disclosure.

FIGS. 6b and 6c show the effect of catechol co-stimulation on the β₂AR ligand-induced DMR signals, in embodiments of the disclosure.

FIG. 7 shows the impact of beta blockers on the catechol response in quiescent A431 cells, in embodiments of the disclosure.

### DETAILED DESCRIPTION

Various embodiments of the disclosure will be described in detail with reference to drawings, if any. Reference to various embodiments does not limit the scope of the invention, which is limited only by the scope of the claims attached hereto. Additionally, any examples set forth in this specification are not limiting and merely set forth some of the many possible embodiments for the claimed invention.

### DEFINITIONS

“Assay,” “assaying” or like terms refers to an analysis to determine, for example, the presence, absence, quantity, extent, kinetics, dynamics, or type of a cell's optical or bioimpedance response upon stimulation with an exogenous stimuli, such as a ligand candidate compound, adenylate activator, cAMP analog, or a GPCR ligand or ligand candidate.

“Attach,” “attachment,” “adhere,” “adhered,” “adherent,” “immobilized”, or like terms generally refer to immobilizing, fixing, culturing, incubating, and like terms, for example, a cell, and like entities of the disclosure, to a surface, such as by physical absorption, chemical bonding, and like processes, or combinations thereof. Particularly, “cell attachment,” “cell adhesion,” or like terms refer to the interacting or binding of cells to a surface, such as by culturing, or interacting with cell anchoring materials, a compatibilizer (e.g., fibronectin, collagen, lamin, gelatin, polylysine, etc.), or both, and like interactions.

“Adherent cells” refers to a cell, a cell line, or a cell system, such as a prokaryotic or eukaryotic cell, that remains associated with, immobilized on, or in certain contact with the outer surface of a substrate. Such cells after culturing can withstand or survive washing and medium exchanging process, which is prerequisite to many cell-based assays. “Weakly adherent cells” refers to a cell, a cell line, or a cell system, such as a prokaryotic or eukaryotic cell, which weakly interacts, associates with or contacts the surface of a substrate during cell culture. However, these types of cells, for example, human embryonic kidney (HEK) cells, tend to dissociate easily from the surface of a substrate by physically disturbing approaches such as washing or medium exchange. “Suspension cells” refers to a cell or a cell line that is preferably cultured in a medium wherein the cells do not attach or adhere to the surface of a substrate during the culture. “Cell culture” or “cell culturing” refer to the process by which either prokaryotic or eukaryotic cells are grown under controlled conditions. “Cell culture” can refer to the culturing of cells derived from multicellular eukaryotes, especially animal cells, and to culturing of complex tissues and organs.

“Cell” or like term refers to a small usually microscopic mass of protoplasm bounded externally by a semipermeable membrane, optionally including one or more nuclei and various other organelles, capable alone or by interacting with other like masses of performing all the fundamental functions of life, and forming the smallest structural unit of living matter capable of functioning independently including synthetic cell constructs, cell model systems, and like artificial cellular systems.

“Cell system” or like term refers to a collection of more than one type of cells (or differentiated forms of a single type of cell), which interact with each other, thus performing a biological or physiological or pathophysiological function. Such cell system includes an organ, a tissue, a stem cell, a differentiated hepatocyte cell, or like cells.

“Marker” or like term refers to a molecule, a biomolecule, or a biological that is able to modulate the activities of at least one cellular target (e.g., a G_{q}-coupled receptor, a Gₛ-coupled receptor, a Gᵢ-coupled receptor, a G_{12/13}-coupled receptor, an ion channel, a receptor tyrosine kinase, a transporter, a sodium-proton exchanger, a nuclear receptor, a cellular kinase, a cellular protein, etc.), and result in a reliably detectable biosensor output or response as measured by a biosensor. Depending on the class of the intended cellular target and its subsequent cellular event(s), a marker could be an activator, such as an agonist, a partial agonist, an inverse agonist, for example, for a GPCR, a receptor tyrosine kinase, an ion channel, a nuclear receptor, or a cellular enzyme adenylate cyclase. The marker could also be an inhibitor for certain classes of cellular targets, for example, an inhibitor or a disruptor for actin filament, or microtubule.

“Detect” or like terms refer to an ability of the system and methods of the disclosure to discover or sense a signaling pathway and to distinguish the sensed signaling pathway from an absence of pathway signaling.

“Identify” or like terms refer to an ability of the system and methods of the disclosure to detect and elucidate a signaling pathway.

“Stimulus,” “therapeutic candidate compound,” “therapeutic candidate,” “prophylactic candidate,” “prophylactic agent,” “ligand candidate,” or like terms refer to a molecule or material, naturally occurring or synthetic, which is of interest for its potential to interact with a cell or a pathogen attached to the biosensor. A therapeutic or prophylactic candidate can include, for example, a chemical compound, a biological molecule, a peptide, a protein, a biological sample, a drug candidate small molecule, a drug candidate biologic molecule, a drug candidate small molecule-biologic conjugate, and like materials or molecular entity, or combinations thereof, which can specifically bind to or interact with at least one of a cellular target or a pathogen target such as a protein, DNA, RNA, an ion, a lipid or like structure or component of a living cell or a pathogen.

“Biosensor” or a like term refers to a device for the detection of an analyte that combines a biological component with a physicochemical detector component. The biosensor typically consists of three parts: a biological component or element (such as tissue, microorganism, pathogen, cells, or combinations thereof), a detector element (operating in a physicochemical way such as optical, piezoelectric, electrochemical, thermometric, or magnetic), and a transducer associated with both components. The biological component or element can be, for example, a live-cell. In embodiments, an optical biosensor can comprise an optical transducer for converting a molecular recognition or molecular stimulation event in a living-cell into a quantifiable signal.

“Include,” “includes,” or like terms means including but not limited to.

“About” modifying, for example, the quantity of an ingredient in a composition, concentrations, volumes, process temperature, process time, yields, flow rates, pressures, and like values, and ranges thereof, employed in describing the embodiments of the disclosure, refers to variation in the numerical quantity that can occur, for example, through typical measuring and handling procedures used for making compounds, compositions, concentrates or use formulations, or in making measurements; through inadvertent error in these procedures; through differences in the manufacture, source, or purity of starting materials or ingredients used to carry out the methods; and like considerations. The term “about” also encompasses amounts that differ due to aging of a composition or formulation with a particular initial concentration or mixture, and amounts that differ due to mixing or processing a composition or formulation with a particular initial concentration or mixture. Whether modified by the term “about” the claims appended hereto include equivalents to these quantities.

“Consisting essentially of” in embodiments refers, for example, to a method for determining ligand-directed functional selectivity for screening pathway-biased or activity-biased ligands in a live-cell as defined herein; a formulation or a composition on the surface of the biosensor; and articles, devices, or apparatus of the disclosure, and can include the components or steps listed or contemplated in the claim(s), plus other components or steps that do not materially affect the basic and novel properties of the compositions, articles, apparatus, and methods of making and use of the disclosure, such as particular reactants, particular additives or ingredients, a particular agent, a particular cell or cell line, a particular surface modifier or surface condition, a particular ligand candidate, or like structure, material, or process variable selected. Items that may materially affect the basic properties of the components or steps of the disclosure or may impart undesirable characteristics to the present disclosure include, for example, decreased affinity of the cell for the biosensor surface, anomalous or contrary cell activity in response to a ligand candidate or like stimulus, and like characteristics.

The indefinite article “a” or “an” and its corresponding definite article “the” as used herein means at least one, or one or more, unless specified otherwise.

Abbreviations, which are well known to one of ordinary skill in the art, may be used (e.g., “h” or “hr” for hour or hours, “g” or “gm” for gram(s), “mL” for milliliters, and “rt” for room temperature, “nm” for nanometers, and like abbreviations).

Specific and preferred values disclosed for components, ingredients, additives, cell types, and like aspects, and ranges thereof, are for illustration only; they do not exclude other defined values or other values within defined ranges. The compositions, apparatus, and methods of the disclosure include those having any value or any combination of the values, specific values, more specific values, and preferred values described herein.

In embodiments the disclosure provides a method for determining ligand-directed functional selectivity of a receptor of a live-cell, the method comprising:

immobilizing a live-cell having a selected receptor on a biosensor;

contacting the immobilized live-cell with a molecular fragment of a ligand of the receptor;

contacting the molecular fragment contacted immobilized live-cell with a stimulus; and

detecting and comparing the stimulus-induced biosensor response of the live-cell in the presence and absence of the molecular fragment.

In embodiments the disclosure provides a method for determining ligand-directed functional selectivity for screening biased ligands in a live-cell, the method comprising:

providing a biosensor having a live-cell immobilized on the biosensor's surface;

contacting the immobilized cell with a molecular fragment (i.e., a ligand fragment) of a receptor ligand;

contacting the molecular fragment treated immobilized cell having a selected receptor with a compound (i.e., stimulus);

detecting the biosensor's output signal in the immobilized live-cell; and

comparing the biosensor's output signal of cell-signaling of the receptor in the presence and absence of the ligand fragment.

In embodiments the fragment contacting and the stimulus compound contacting can be accomplished, for example, simultaneously, sequentially, or in inverse order. In embodiments “a receptor of a live-cell” refers to, for example, a receptor (including artificial or natural constructs) associated with a particular live-cell. The association of the receptor with the live-cell can be, for example, internal or within the cell, external, such as on the cell surface, a transmembrane disposition, and like cellular dispositions, or a combination thereof,

In embodiments the molecular fragment can be, for example, a synthetic or naturally occurring structural component of a natural ligand of the receptor, or can be, for example, a synthetic or naturally occurring partial sequence of a natural ligand if the ligand is, for example, a peptide or protein. In embodiments the receptor can be, for example, a G protein-coupled receptor (GPCR). In embodiments the GPCR can be, for example, a β2-adrenergic receptor (β₂AR), and the ligand fragment can be, for example, catechol or halostachine. In embodiments the biosensor's output signal can be, for example, the dynamics, the phases, the signal amplitude, the kinetics of each phase, the transition time from one phase to another phase, or a combination thereof, and like output phenomena.

In embodiments the disclosure provides methods to examine ligand-directed functional selectivity using label-free biosensors. The method provides an integrated cellular response, in combination with a fragment of a natural ligand for a given G protein coupled receptor (GPCR) as a molecular probe. The biosensor's output signal can be, for example, a dynamic mass redistribution signal if an optical biosensor is used, or a bioimpedance signal if an electrical biosensor is used. Using the β₂AR as an example model system, a multi-parameter analysis of agonist-induced DMR signals identified unexpected patterns in activation and signaling of the β₂AR. Cross-examination with various combinations of β₂AR ligands for stimulation resulted in unexpected patterns of ligands to modulate cell signaling induced by catechol, a structural component of catecholamine agonists. In embodiments, a full agonist, epinephrine, was able to override the catechol response, and as did the strong partial agonist norepinephrine. Conversely, an antagonist, including betaxolol, and an inverse agonist, including ICI 118551, dose-dependently attenuated the catechol response. A partial agonist, such as alprenolol or pindolol that are not effective in causing cAMP accumulation, lead to signaling that was mostly independent of the catechol response. These results exemplify multiple ligand-specific states of the β₂AR, and demonstrate the utility of a measurable integrated cellular response such as DMR for screening biased ligands.

In embodiments the disclosure provides a method for molecular fragment-based functional screening against a receptor of a live-cell using label-free biosensor, the method comprising:

immobilizing a live-cell having a selected receptor on a biosensor;

contacting the immobilized live-cell with a first molecular fragment;

contacting the immobilized live-cell with a second molecular fragment; and

detecting and comparing the molecular fragment-induced biosensor response of the live-cells.

In embodiments of the screening method the contacting with the first molecular fragment and contacting with the second molecular fragment can be accomplished for example, simultaneously, sequentially, or in reverse order.

G protein-coupled receptors (GPCRs) are the largest family of cell membrane receptors in the human genome and the richest class of drug targets. The primary function of GPCRs as signaling molecules is to transduce exogenous information into intracellular signals. In classical models of drug action, the primary event is binding of a ligand to its receptor, which, in turn, leads to a cellular effect whose magnitude depends on the intrinsic efficacy of the ligand. Classical receptor-occupancy theory defines the efficacy of ligands as their ability to alter the equilibrium between inactive and active states of the receptor, assuming that all GPCR activities are correlated. However, the data collected in recent decades have challenged this simple kinetic model. More recent evidence suggests that GPCR signaling is highly complex and sophisticated. For example, a receptor may couple simultaneously to more than one G protein subtype, and interact with other signaling molecules and scaffolding proteins such as arresting. In many instances the activation of a receptor can mediate both G protein-dependent and independent signaling, often in a ligand-dependent manner. As a result, GPCRs display rich behaviors in cell systems, and many ligands can induce operative bias to favor specific portions of the cell machinery and exhibit pathway-biased activities and efficacies. The complexity of G protein coupled receptor (GPCR) signaling and ligand-directed functional selectivity calls for high resolution tools for studying GPCR behavior.

Pathway biased activities of ligands have been illustrated in many types of GPCRs including the β₂-adrenergic receptor (β₂AR). The underlying mechanism for multiplicity of efficacies has been attributed to the ability of compounds to promote unique, ligand-selective conformations of receptors that have differential propensities to engage different pathways. However, direct links between specific receptor conformations and differential signaling efficacies have heretofore not been clearly established.

The existence of pathway having biased efficacies for a ligand through a unique receptor may indicate that classifying compounds on the basis of their ability to modulate a single effector system is not sufficient for a complete description of their signaling potentials. Given the significant implications of ligand-directed functional selectivity in drug development, the possibility of a ligand having multiple efficacies creates considerable demand in high resolution pharmacological assays for studying GPCR behaviors and screening of biased ligands. Although resolution has been improving in recent years, most of the conventional cell assays only monitor cellular events associated with a single signaling pathway. Therefore, a multitude of assays may be needed to differentiate the functional consequences of distinct ligands. Furthermore, since the GPCR signaling is dependent on the cellular background, the use of engineered cell systems, which are commonly a prerequisite for many conventional approaches, can complicate the assignment of ligand-directed functional activity.

Fragment-based screening is a method that can be used for developing, for example, novel lead compounds. The goal is to identify smaller, lower molecular weight fragments that bind with high efficiency in an active site. Smaller fragments have “room-to-grow” in that they can, for example, be linked-together or built-up, for example, to generate novel high affinity compounds within the size constraints of a typical drug molecule. Paradoxically, fragment-based drug design is based on screening smaller numbers of compounds with an expectation of finding low-affinity fragments, such as with K_{d} values in the high micromolar to millimolar range, using bio-molecular binding assays, including two-dimensional, isotope-edited nuclear magnetic resonance (NMR) spectroscopy assays, X-ray crystal binding assays, Surface Plasmon Resonance (SPR) binding assays, or like methods. Conceptual fragmentation of an active drug compound produces smaller pieces, or even discrete functional groups (for example, carboxylate, amine, aryl, and like groups). Such a technique has been used for some time to simplify the computational analysis of ligand binding and to map out different pharmacophoric elements required for high-affinity binding. The technique is simple in that proper optimization of each unique interaction in the binding site and subsequent incorporation into a single molecular entity should produce a compound with a binding affinity that is the sum of the individual interactions. The present disclosure describes functional cell-based assays that can be applied to fragment-based screening, specifically in living cells. Instead of binding, the disclosed method involves the use of label-free biosensor to detect ligand-directed functional selectivity in live-cells. This method is based on a hypothesis that a selected ligand fragment can result in a subset of cell-signaling that is mediated through a receptor by its natural ligand.

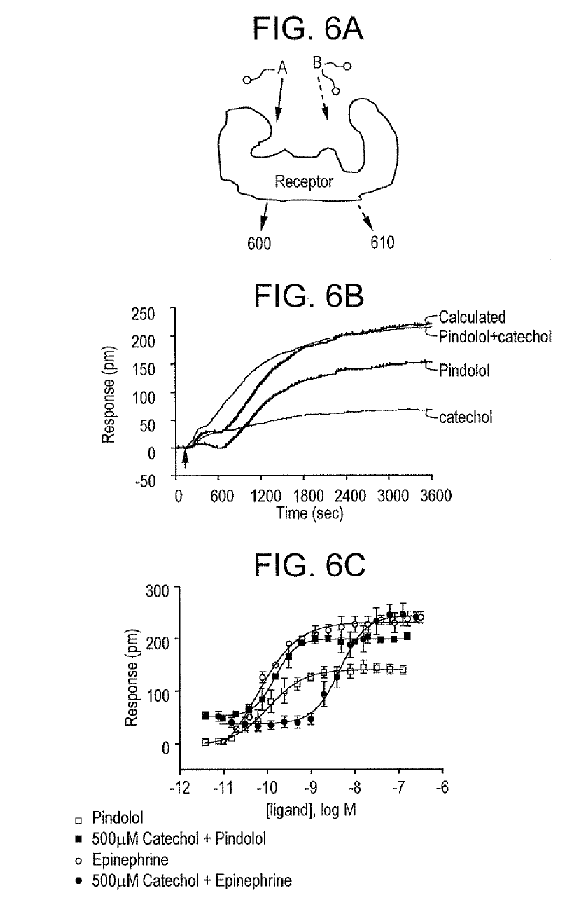

FIG. 6a shows a hypothetical situation wherein two ligands or ligand fragments (A and B) can bind to but recognize different binding sites, and thus each independently mediates different sets of cell signaling (600 or 610, respectively). The signaling of 600 or 610 can be completely separated, or can partially overlap. Assaying the functional selectivity of ligands, ligand fragments, or both, using label-free biosensor methodology, can identify the mode(s) of action of each ligand or ligand fragment, and the mode(s) of action of co-stimulation of both ligands or ligand fragments. The resulting ligand-directed functional selectivity can be used to guide fragment design and selection, for example, to make drug leads or to diagnose defective metabolic pathways or processes.

Optical biosensors can measure ligand-induced cellular responses of cells by detecting minute changes in local mass density or mass redistribution in the cells. Thus, optical biosensor-based live-cell assays do not require prior knowledge of cell signaling, and can enable direct measurement of ligand-induced receptor activation and signaling. This is in contrast to most conventional technologies that may rely on the measurement of ligand-induced biological responses linked to a single signaling pathway. Since a receptor is not necessarily faithful to a single signaling pathway, these conventional assays can lead to false negatives.

A ligand-induced DMR signal measured with an optical biosensor provides a method to study ligand-directed trafficking of receptor signaling (i.e., ligand-selective signaling, or ligand-directed functional selectivity). The DMR can approximately globally represent receptor signaling, provide real time kinetic information, and have sensitivity to signaling pathways. Beside its dynamics and signal amplitudes, the kinetic parameters of a ligand-induced DMR signal provide for analyzing receptor signaling and ligand-biased potencies and efficacies. Studies have shown that a ligand acting on a single receptor may vary in its ability to modulate one pathway over another, leading to multitude of potential activities. Due to the large number of possible signaling outputs, it can be difficult to systematically represent the efficacy of a ligand-receptor pair using conventional pathway-biased approaches.

In addition to potential false negatives due to the pathway bias, conventional technologies require some manipulation or engineering to boost assay sensitivity and resolution. These manipulations can create a heterologous expression system and increase the propensity of the over-expressed receptors to interact with distinct G proteins, and may lead to misleading and promiscuous behavior of GPCRs. In contrast, optical biosensors offer a non-invasive and manipulation-free alternative method to assay endogenous GPCRs in native cells.

### Label-Free Biosensor-Based Cell Assays

Label-free cell-based assays generally employ a biosensor to monitor ligand-induced responses in living cells. A biosensor typically utilizes a transducer such as an optical, electrical, calorimetric, acoustic, or magnetic transducer, to convert a molecular recognition event or a ligand-induced change in a cell layer into a quantifiable signal. These label-free biosensors are commonly used for molecular interaction analysis, which involves characterizing how molecular complexes form and disassociate over time. There are two types of biosensors that can be used for label-free cell-based assays—resonant waveguide grating (RWG) biosensors and electrical biosensors.

RWG biosensors and systems—An RWG biosensor consists of a substrate (e.g., glass), a waveguide thin film with an embedded grating structure, and a cell layer. The RWG biosensor utilizes the resonant coupling of light into a waveguide by means of a diffraction grating, leading to total internal reflection at the solution-surface interface, which in turn creates an electromagnetic field at the interface. This electromagnetic field is evanescent and decays exponentially from the sensor surface; the distance at which it decays to 1/e of its initial value is known as the penetration depth and is a function of the design of a particular RWG biosensor, but is typically on the order of about 200 nm. This type of biosensor exploits evanescent waves to characterize ligand-induced alterations of a cell or cell layer at or near the sensor surface.

RWG instruments can be subdivided into systems based on angle-shift or wavelength-shift measurements. In a wavelength-shift measurement, polarized light covering a range of incident wavelengths with a constant angle is used to illuminate the waveguide; light at specific wavelengths is coupled into and propagates along the waveguide. Alternatively, in angle-shift instruments, the sensor is illuminated with monochromatic light and the angle at which the light is resonantly coupled is measured. The resonance conditions are influenced by the cell layer (e.g., cell confluency, adhesion, status, etc.), which is in direct contact with the surface of the biosensor. When a ligand or an analyte interacts with a cellular target (e.g., a GPCR, a kinase, etc.) in living cells, any change in local refractive index within the cell layer can be detected as a shift in resonant angle (or wavelength).

The Corning® Epic® system uses RWG biosensors for label-free biochemical or cell-based assays (Corning Inc., Corning, N.Y.). The Epic® System consists of an RWG plate reader and SBS standard microtiter plates. The detector system in the plate reader exploits integrated fiber optics to measure the shift in wavelength of the incident light as a result of ligand-induced changes in the cells. A series of illumination/detection heads are arranged in a linear fashion so that reflection spectra are collected simultaneously from each well within a column of a 384-well microplate. The whole plate can be scanned so that each sensor can be addressed multiple times, and each column is addressed in sequence. The wavelengths of the incident light are collected and used for analysis. A temperature-controlling unit can be built in the instrument to minimize spurious shifts in the incident wavelength due to the temperature fluctuations.

Electrical biosensors and systems—Electrical biosensors consist of a substrate (e.g., plastic), an electrode, and a cell layer. In this electrical detection method, cells are cultured on small gold electrodes arrayed onto a substrate, and the system's electrical impedance is followed with time. The impedance is a measure of changes in the electrical conductivity of the cell layer. Typically, a small constant voltage at a fixed frequency or varied frequencies is applied to the electrode or electrode array, and the electrical current through the circuit is monitored over time. The ligand-induced change in electrical current provides a measure of cell response. Impedance whole-cell sensing measurements were first achieved in 1984. Since then impedance-based measurements have been applied to study a wide range of cellular events, including cell adhesion and spreading, cell micromotion, cell morphological changes, and cell death. Such impedance systems suffer from high assay variability due to use of a small detection electrode and a large reference electrode. To overcome this variability, the modern systems, such as the CellKey system (MDS Sciex, South San Francisco, Calif.) and RT-CES (ACEA Biosciences Inc., San Diego, Calif.), utilize an integrated circuit having a microelectrode array.

The CellKey system consists of an environmentally controlled impedance measurement system, a 96-well electrode-embedded microtiter plate, an onboard 96-well fluidics, and acquisition and analysis software. The cells are seeded in the culture wells; each well has an integrated electrode array. The system operates using a small-amplitude alternating voltage at 24 frequencies, from 1 KHz to 10 MHz. The resultant current is measured at an update rate of 2 sec. The system is thermally regulated and experiments can be conducted between 28° C. and 37° C. A 96-well head fluid delivery device handles fluid additions and fluid exchanges onboard.

The RT-CES system is composed of four main components: electronic microtiter plates (E-Plate™), E-Plate station, electronic analyzer, and a monitoring system for data acquisition and display. The electronic analyzer sends and receives the electronic signals. The E-Plate station is placed inside a tissue culture incubator. The E-Plate station comes in three throughput varieties: a 16× station for running six 16-well E-Plates at a time, a single 96-well E-Plate station, and the Mult-E-Plate™ station, which can accommodate up to six 96-well E-Plates at a time. The cells are seeded in E-Plates, which are integrated with microelectronic sensor arrays. The system operates at a low-voltage (less than 20 mV) AC signal at multiple frequencies.

Optical signals of GPCR activation with RWG biosensor—Cells are dynamic objects with relatively large dimensions—typically tens of microns. RWG biosensors enable detection of ligand-induced changes within the portion of cells closest to the sensor surface, determined by the penetration depth of the evanescent wave. Furthermore, the spatial resolution of an optical biosensor is determined by the spot size (about 100 microns) of the incident light source. Thus, although low confluent cells can be used for assaying, a highly confluent cell layer is generally used in order to achieve optimal assay results. The sensor configuration can be viewed as a three-layer waveguide composite consisting of, for example, a substrate, waveguide thin film, and a cell layer. We have discovered that for whole-cell sensing, a ligand-induced change in effective refractive index, the detected signal ΔN, is governed by eq. (1):

$$\begin{matrix}Δ N = S ( N ) Δ {n}_{c} \\ = S ( N ) α d {\sum }_{i} Δ {C}_{i} [ {e}^{\frac{- {z}_{i}}{Δ {Z}_{C}}} - {e}^{\frac{- {z}_{i + 1}}{Δ {Z}_{C}}} ]\end{matrix} \qquad ( 1 )$$

where S(C) is the system sensitivity to the cell layer, and Δn_{c} is the ligand-induced change in local refractive index of the cell layer sensed by the biosensor. ΔZ_{c} is the penetration depth into the cell layer, α is the specific refractive index increment (about 0.18/mL/g for proteins), zᵢ is the distance where the mass redistribution occurs, and d is an imaginary thickness of a slice within the cell layer. Here the cell layer is divided into an equally-spaced slice in the vertical direction. The detected signal is assumed to be to first order, directly proportional to the change in refractive index of the bottom portion of cell layer Δn_{c}. Δn_{c} in turn is directly proportional to changes in local concentration of cellular targets or molecular assemblies within the sensing volume, given the refractive index of a given volume within cells is largely determined by the concentration of bio-molecules, mainly proteins. A weighted factor exp(−zᵢ/ΔZ_{c}) is taken into account for a change in local protein concentration that occurs, considering the exponentially decaying nature of the evanescent wave. Thus, the detected signal is the sum of mass redistribution occurring at distinct distances away from the sensor surface, each with unequal contribution to the overall response. Eq. 1 suggests that the detected signal with an RWG biosensor is sensitive primarily to the vertical mass redistribution, as a result of a change in local protein concentration. The detected signal is often referred to as a dynamic mass redistribution (DMR) signal. GPCR activation leads to a series of spatial and temporal events, including, for example, ligand binding, receptor activation, protein recruitment, receptor internalization and recycling, second messenger alternation, cytoskeletal remodeling, gene expression, and cell adhesion changes. Each cellular event has its own characteristics regarding its kinetics, duration, amplitude, and mass movement. Thus it is reasonable to assume that these cellular events may contribute differently to the overall DMR signal, depending on where they occur. Using a panel of agonists targeting a variety of GPCRs, three classes of DMR signals were identified in human epidermoid carcinoma A431 cells, which reflect the signaling pathways mediated. Since each is correlated with the activation of a class of GPCRs depending on the G protein with which the receptor is coupled, the DMR signals obtained were named G_{q}-, Gₛ- and Gᵢ-DMR signals, respectively. Each class of DMR signals exhibits distinct kinetic and dynamic characteristics, reflecting the unique signaling integration mediated through different classes of GPCRs. The unique characteristics of the DMR signals can be used to identify the G-protein coupling mechanism of orphan GPCRs.

Bioimpedance signals of GPCR activation—In a typical impedance-based cell assay, cells are brought into contact with a gold electrode arrayed on the bottom of culture wells. The total impedance of the sensor system is determined primarily by the ion environment surrounding the biosensor. Under application of an electrical field, the ions undergo field-directed movement and concentration gradient-driven diffusion. For whole cell sensing, the total electrical impedance has four components: the resistance of the electrolyte solution, the impedance of the cell, the impedance at the electrode/solution interface, and the impedance at the electrode/cell interface. In addition, the impedance of a cell comprises two components—the resistance and the reactance. The conductive characteristics of cellular ionic strength provide the resistive component, whereas the cell membranes, acting as imperfect capacitors, contribute a frequency-dependent reactive component. Thus, the total impedance is a function of many factors, including cell viability, cell confluency, cell numbers, cell morphology, degree of cell adhesion, ionic environment, the water content within the cells, and the detection frequency.

In the RT-CES system, a percentage of this small voltage applied is coupled into the cell interior. Such signals applied to cells are believed to be much smaller than the resting membrane potential of a typical mammalian cell and thus present minimal or no disturbance to cell function. The RT-CES system measures these changes in impedance and displays it as a parameter called the cell index. The cell index is calculated according to the formula of eq. (2):

$$CI = {max}_{i = 1 , … , N} ( \frac{{R}_{cell} ( {f}_{i} )}{{R}_{0} ( {f}_{i} )} - 1 ) \qquad ( 2 )$$

where N is the number of frequency points at which the impedance is measured (e.g., N=3 for 10 kHz, 25 kHz, and 50 kHz), and R₀(f) and R_{cell}(f) are the frequency electrode resistance without cells or with cells present in the wells, respectively.

In the CellKey system, a change in sensor system's impedance is attributed to a change in complex impedance (delta Z or dZ) of a cell layer that occurs in response to receptor stimulation. At low frequencies, the small voltage applied induces extracellular currents (iec) that pass around individual cells in the layer. However, the conduction currents through cell membrane due to ion channels may also be important at low measurement frequencies. At high frequencies, they induce transcellular currents (itc) that can penetrate the cell membrane. The ratio of the applied voltage to the measured current for each well is its impedance (Z) as described by Ohm's law.

When cells are exposed to a stimulus, such as a receptor ligand, signal transduction events are activated that lead to complex cellular events such as modulation of the actin cytoskeleton that can cause changes in cell adherence, cell shape and volume, and cell-to-cell interaction. These cellular changes individually or collectively affect the flow of extracellular and transcellular current, and therefore, affect the magnitude and characteristics of the measured impedance. At least three types of impedance signals can be mediated through the activation of three classes of GPCRs, depending on the G protein to which the receptor is coupled. The impedance profiles can be obtained using, for example, a CellKey system (www.cellkey.com). Similar profiles can be recorded using the RT-CES system. Although not limited by theory it is believed that these impedance signals are due to the different effects on the actin cytoskeleton that affect the cellular parameters measured by impedance, in response to the activation of different classes of GPCRs. The activation of G_{q} and Gᵢ GPCRs has been shown to lead to increased actin polymerization, while stimulation of Gₛ GPCRs leads to actin depolymerization.

### EXAMPLES

The following examples serve to more fully describe the manner of using the above-described disclosure, and to further set forth the best modes contemplated for carrying out various aspects of the disclosure. These examples do not limit the scope of this disclosure, but rather are presented for illustrative and demonstrative purposes.

The disclosed label-free biosensor methods follow stimulus-induced cellular responses which are integrated or aggregate responses. The methods can enable and accomplish ligand-induced functional selectivity determinations.

### Example 1

### Functional Diversity and Ligand-Directed Functional Selectivity

Pathway biased activities of ligands have been illustrated in β₂-adrenergic receptor (β₂AR). An excellent example is that ICI 118551 and propranolol are inverse agonists for Gₛ-stimulated adenylyl cyclase, but are partial agonists for the mitogen-activated protein kinases (MAPKs) extracellular signal-regulated kinase (ERK1/2) in HEK293 fibroblasts stably expressing the β₂AR (Azzi, M., et. al., Proc. Natl. Acad. Sci. U.S.A., 100, 11406-11411 (2003)). The β₂AR is a prototypical family A receptor activated by catecholamines, whose interaction sites with the receptor are mostly known. Several recent biophysical studies showed that for the β₂AR ligand binding and activation is a kinetically and conformationally complex process, and agonist binding and conformational changes occur through a sequence of conformational intermediates. Notable conformational changes include the disruption of an ionic-lock consisting of the (D/E)R(W/Y) sequence at the cytoplasmic end of transmembrane helix 3 (TM3) and an acidic amino acid at the cytoplasmic end of TM6, and the activation of a “rotamer toggle switch” involving a change in the bend of TM6 at the highly conserved proline kink at Pro288^{6.50}. The two switches can be activated independently of each other, and agonists may have different abilities to activate them. However, additional switches exist and must be activated to achieve the fully active state of the receptor. These findings raise questions about the molecular basis of signaling efficacy. The underlying mechanism for multiplicity of efficacies has been attributed to the ability of compounds to promote unique, ligand-selective conformations of receptors that have differential propensities to engage different pathways. However, direct links between specific receptor conformations and differential signaling efficacies have not been established.

### EXPERIMENTAL PROCEDURES

Materials—Alprenolol, a cell preamble dynamin inhibitory peptide control (DIPC), CGP 12177, dopamine, epinephrine, forskolin, ICI 118551, isoproterenol, labetalol, norepinephrine, pindolol, S(−)pindolol, propranolol, salbutamol, salmeterol, timolol, and xamoterol were obtained from Tocris (St. Louis, Mo.). Catechol, halostachine, tyramine, and phenylethylamine were purchased from Sigma Chemical Co. (St. Louis, Mo.). Epic® 384well cell assay microplates were obtained from Corning Inc (Corning, N.Y.). The microplates, in which each well consists of a resonant waveguide grating (RWG) biosensor, are ready-to-culture and were used directly without any pretreatments.

Cell culture—Human epidermoid carcinoma A431 cells (American Type Cell Culture) were grown in Dulbecco's modified Eagle's medium (DMEM) supplemented with 10% fetal bovine serum (FBS), 4.5 g/liter glucose, 2 mM glutamine, and antibiotics. About 1.8×10⁴ cells at passage 3 to 8 suspended in 50 μL the DMEM medium containing 10% FBS were placed in each well of a 384-well microplate, and were cultured at 37° C. under air/5% CO₂ for about 1 day, followed by about 20 hr starvation through continuously culturing in the serum-free DMEM.

Optical biosensor system and cell assays—A Corning® Epic® wavelength interrogation system with an on-board liquid handling system was used. The system is standalone and consists of a temperature-control unit, an optical detection unit, and an on-board liquid handling unit with robotics. The temperature-control unit minimizes temperature fluctuations, if any. Inside this unit, there are two side-by-side stacks for holding both the sensor microplates and compound source plates. Once stable temperature is reached (typically within 1 hr), a sensor microplate is transferred by the robotics into the plate holder right above the detection system, while a source plate is moved to an appropriate compartment so that it is readily addressable by the on-board liquid handling unit.

The detection unit has integrated fiber optics to measure the wavelength shift of the resonant lights due to ligand-induced dynamic mass redistribution in living cells. A broadband white light source centered on 830 nm, generated through a fiber optic and a collimating lens at nominally normal incidence through the bottom of the microplate, is used to illuminate a small region of the grating surface. A detection fiber for recording the reflected light is bundled with the illumination fiber. A series of illumination/detection heads are arranged in a linear fashion, so that reflection spectra are collected from the 16-wells within the same column of a 384 well microplate at once. The whole plate is scanned by the illumination/detection heads so that each sensor can be addressed multiple times, and each column is addressed in sequence, leading to a kinetic measurement of cellular responses with a time interval of 6 or 13 sec.

For kinetic assays, the cells were washed with HBSS (Hanks Balanced Salt Solution with 20 mM HEPES) buffer. After 1 hour incubation within the detection system, the sensor plate was scanned and a baseline response was recorded. Then, compound solutions were transferred into the sensor plate using the on-board liquid handling system, and the cell responses were then recorded for another period of time. The lid of the sensor microplates was on throughout the assay, except of a short period of time (about 1 min.) when the compounds were introduced. The plate lid was handled automatically by the robotics. All studies were carried out at controlled temperature (28° C.).

Statistical analysis—Unless specifically mentioned, three replicates were carried out for each measurement or each compound. The standard deviation was derived from these measurements (n=3). The assay coefficient of variation was found to be typically less than about 10%. All dose-dependent responses were analyzed using non-linear regression method with Prism software (available from Graph Pad).

### Results

Ligand-specific DMR signals—β₂AR in A431 cells were selected as a model system for assaying ligand-directed functional selectivity, because of well-established signaling pathways of the β₂AR in A431 as well as the availability of a rich source of structurally similar ligands having wide spectrum of efficacies. A431 cells endogenously express large numbers of the β₂AR (about 34,000 copies per cells). To examine the functional selectivity of β₂AR ligands, we used RWG biosensor to follow change in local mass density or distribution as a function of time upon stimulation with a saturating concentration of a ligand. The RWG biosensor detects an integrated response, termed as dynamic mass redistribution (DMR) signal, mediated through ligand-induced activation of the β₂AR. The DMR signal approximates a global representation of the receptor activation and signaling, particularly for signaling events associated with dynamic relocation of cellular matters at or near the sensor surface.

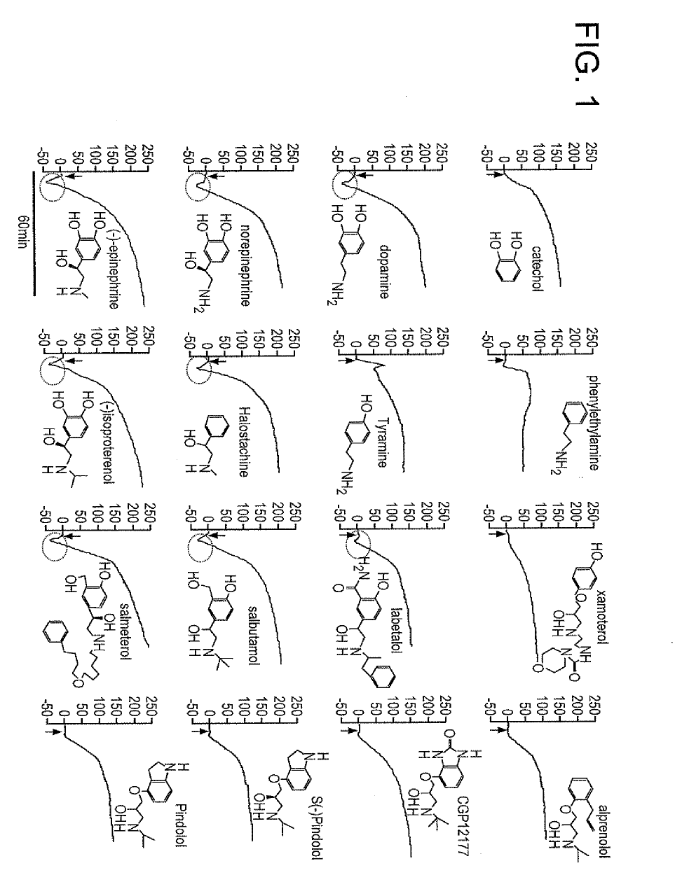

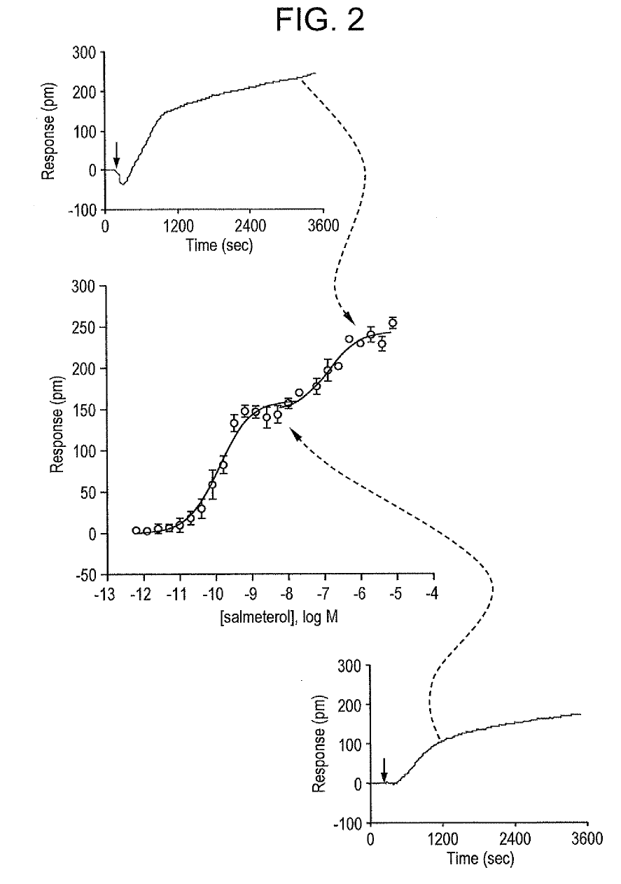

FIG. 1 shows characteristic DMR signals of quiescent A431 cells upon stimulation with an array of β₂AR ligands, and the chemical structures of these ligands. The ligands included (−)epinephrine (8 nM), (−)isoproterenol (10 nM), norepinephrine (100 nM), dopamine (32 μM), halostachine (500 μM), catechol (500 μM), tyramine (125 μM), phenylethylamine (500 μM), salmeterol (8,000 nM), salbutamol (164 nM), labetalol (2 μM), xamoterol (1 μM), pindolol (8 μM), S(−)pindolol (8 μM), CGP12177 (100 nM), and alprenolol (4 μM). Each arrow indicates when respective agonist solutions were introduced on a 60 minute time scale. Table 1 summarizes the parameters (affinity, potency, efficacy, and kinetics) of ligand-induced DMR signals. As shown in FIG. 1, all signals started with a short baseline (about 2 min.). The DMR signals induced by these β₂AR ligands can be classified into at least five distinct categories, based on the dynamics of the DMR signals. The first group of ligands included isoproterenol, epinephrine, norepinephrine, dopamine, halostachine, and salbutamol, which at a saturating concentration each mediated a biphasic response of cells. Followed a small decrease signal (termed as negative-DMR, N-DMR) with a short duration was an increased signal (termed as positive-DMR, P-DMR) to an elevated level with a ligand-dependent maximal amplitude and kinetics. Labetalol at a saturating concentration also resulted in a biphasic response. However, its initial N-DMR was much less evident. The second group of ligands included catechol, xamoterol, alprenolol, CGP12177, S(−) pindolol, and pindolol. These ligands at a saturating concentration resulted in an initial steady phase (termed as net-zero DMR) with a short duration, and a succeeding P-DMR. The third group of ligands included salmeterol. Salmeterol led to a biphasic dose-dependent DMR signal. FIG. 2 shows a dose-dependent DMR response induced by salmeterol, as plotted as the amplitude of the P-DMR event as a function of salmeterol concentration in A431 cells. FIG. 2 also shows a combination of representative DMR signals induced by salmeterol at two different doses: 10 nM (top insert) and 1,000 nM (bottom insert), respectively. At low doses (<100 nM) salmeterol led to a DMR signal similar to that induced by the second group of ligands such as CGP 12177. However, at high doses the salmeterol-induced DMR signal was similar to that induced by epinephrine. The fourth group of ligands included phenylethylamine and tyramine. Both ligands led to a complicated DMR signal, possibly due to co-activation of other classes of receptors in addition to the β₂AR (data not shown). Because of their complexity, these two ligands were excluded from further analysis. The fifth group of ligands included ICI 118551, propranolol, timolol, satolol, betaxolol, atenolol, and CGP20712, none of which led to any significant DMR signal (data not shown). Given the sensitivity of the DMR signals to signaling pathway involved, these results suggest that ligands differ greatly in their ability to activate the β₂AR and direct its cellular signaling.

Ligand-induced DMR signals are specific to the β₂AR—Since A431 cells also endogenously express other receptors including Gₛ-coupled receptors, we sought to discover whether the DMR signals obtained are specific to the β₂AR or not. Results showed that propranolol completely dose-dependently attenuated the DMR signals induced by all agonists at a concentration of about EC₁₀₀, except for catechol as discussed below and summarized in Table 2. The IC₅₀ obtained was within 1 to 10 nM, except for salmeterol of 100 nM for which the IC₅₀ of propranolol was about 58 nM. Interestingly, dopamine of <100 μM led to a saturable response that can be completely inhibited by 1 μM propranolol, but neither 1 μM SCH 23390 nor 1 μM spiperone (both are dopamine receptor antagonists). However, dopamine at high doses (>100 μM) led to a new type of DMR signal that can be only partially inhibited by 1 μM propranolol. Subtracting the dopamine response of the propranolol-treated cells from the dopamine response in cells without any pretreatment led to a DMR signal that closely resembled the DMR signal induced by 32 μM dopamine alone (data not shown), suggesting that dopamine at high doses can cross-talk with other receptors in A431 cells. Thus, results and discussion were limited to dopamine at doses <100 μM. Nonetheless, these results suggest that the DMR signals induced by these ligands are β₂AR-specific.

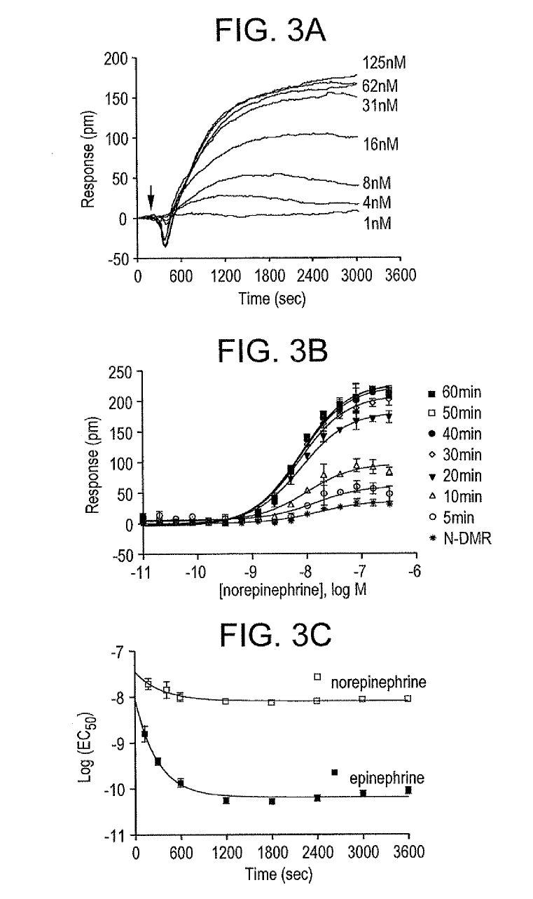

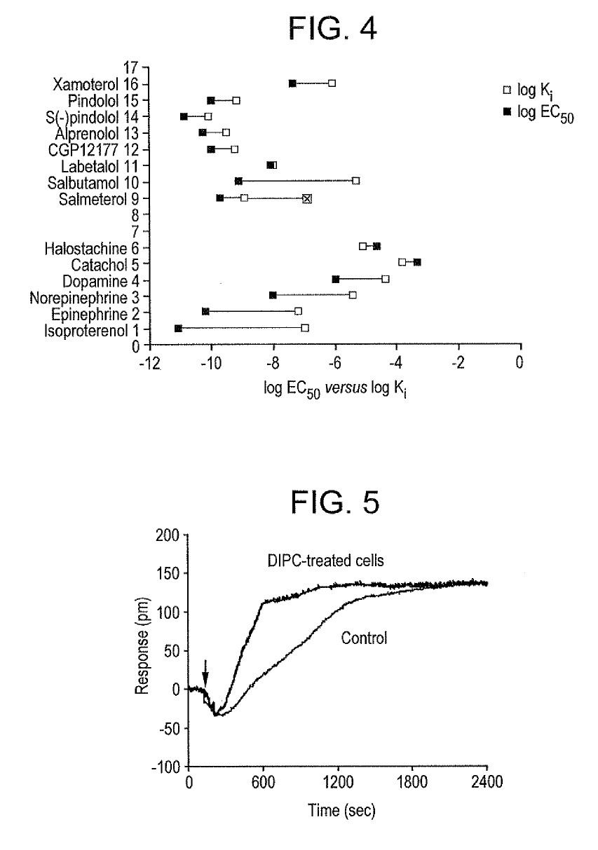

Ligand-specific efficacy—A Mass Redistribution Cell Assay Technology (MRCAT)(see ref. 6, Fang, Y., et al.) assay typically involves the continuous monitoring of cellular responses over a longer period of time (e.g., about 1 hr) after ligand addition. This type of sustained stimulation with an agonist should be taken into account for ligand pharmacological analysis. FIGS. 3a to 3c show the norepinephrine-induced DMR signals as follows: a) dose-dependent DMR signals of quiescent A431 cells; b) amplitudes of DMR signals as a function of assay time; and c) apparent EC₅₀ of norepinephrine as a function of assay time compared with the epinephrine response. FIG. 3a results showed dose-dependent responses of A431 cells induced by norepinephrine. The shifts in resonant wavelength at different time points were calculated by subtracting the minimal wavelength after stimulation from the wavelength at each time point. The resultant wavelength shifts at each time point were plotted as a function of norepinephrine concentrations, all of which clearly displayed dose-dependence and were saturable (FIG. 3b). The EC₅₀ values obtained were found to be somewhat time-dependent, which followed a single-phase exponential decay with a t_{1/2} of about 248 sec (FIG. 3c). When the continuous stimulation was longer than about 10 min, the EC₅₀ values obtained were almost constant. Similar trend was observed for epinephrine, but which displayed more rapid and significant decay with a t_{1/2} of about 203 sec (FIG. 3c). For convenience and assay sensitivity, the time point at 50 min. was chosen to calculate the efficacies and potencies of all β₂AR ligands. FIG. 4 shows a relationship between the logs of apparent EC₅₀ and logs of binding affinity of selected β₂AR agonists.

The maximal amplitudes of the P-DMR events, normalized to the epinephrine response, were found to be in the following order: isoproterenol (105%) about the same as epinephrine (100%)>norepinephrine (91%) about the same as dopamine (92%) about the same as salbutamol (91%) about the same as halostachine (90%)>catechol (66%) about the same as alprenolol (65%) about the same as labetalol (62%) about the same as pindolol (61%) about the same as S(−)pindolol (60%)>CGP 12177 (54%) about the same as alprenolol (54%)>xamoterol (33%) (n=16). Similar trend was observed for the N-DMR events (Table 1). These results suggest that both isoproterenol and epinephrine fully or nearly fully activate the β₂AR, while the other ligands partially activate the receptor. However, the difference in efficacies is much less pronounced than those reported in literature based on the measurements of intracellular cAMP level. This may due to relatively high expression level of the β₂AR in A431 cells, which may cause masking of the partial agonist activity of the compounds tested. Nonetheless, these results suggest that β₂AR ligands that result in the same type of DMR signals may have different efficacies.

Ligand-specific shift of apparent potency relative to affinity—Many studies have shown that, similar to the efficacy, the potency of ligands can vary greatly and can depend upon the signaling pathway being assayed. The shift in apparent potency relative to affinity has been attributed to the differential coupling efficiencies of ligands. Thus, the relationship between the apparent potency and affinity of these ligands was of interest. Results showed that all β₂AR ligands gave rise to a dose-dependent and saturable response, resulting in a single apparent EC₅₀ (data not shown), except that salmeterol exhibited two well-separated EC₅₀ values (0.12±0.05 nM and 130±17 nM) (FIG. 2). The potency determination uncovered significant difference among the shifts in potency of various ligands relative to binding affinity (Table 1 and FIG. 4). Because of its biphasic nature of dose-dependent responses, the potency of salmeterol shifted towards both directions. In comparison, the EC₅₀ of two full agonists, isoproterenol and epinephrine, significantly shifted towards the left. A similar shift was observed for three strong partial agonists, norepinephrine, dopamine, and salbutamol, which are known to be effective in activating Gₛ, leading to slightly less cAMP accumulation than epinephrine. The large observed shift in EC₅₀ may be due to, for example, a relatively low threshold of an elevated cAMP level that causes an epinephrine-like DMR signal, the persistent stimulation in the MRCAT assays, or both. In contrast, the EC₅₀ values of other weak or very weak partial agonists for activating Gₛ were found to be close to their corresponding k_{d}. These results suggest that the weaker the agonist is, the closer the k_{d} will be to EC₅₀; and the greater the shift of the EC₅₀ indicates that the ligand is more effective in causing cAMP accumulation. Thus, in embodiments the label-free methods of the disclosure can be used to characterize the influence of, for example, a stimulus or a therapeutic candidate on the potency and affinity of a ligand for a receptor, and additionally the relationship between the potency and affinity.

Ligand-specific kinetics of DMR signals—The DMR signal is an integrated response, consisting of contributions from many downstream signaling events that involve dynamic relocation of cellular matters within the sensing volume of the biosensor. Thus, a DMR signal contains high information regarding to receptor signaling. Besides their overall dynamics, phases and signal amplitudes, the DMR signals offer additional kinetic information, including the transition time (τ) from one phase to another phase, and the kinetics of each phase. Here the transition time refers to the time for the P-DMR event to occur. As summarized in Table 1, ligands that resulted in rapid transition (τ of about 140 sec) included isoproterenol, epinephrine, catechol, and salbutamol. Except for catechol which is a very weak partial agonist to stimulate Gₛ, the other three ligands are full agonists or strong partial agonists. In comparison, all other ligands including salmeterol of 10 nM resulted in a slow transition (τ of about 200-300 sec). As expected, for salmeterol the transition time was dependent on the salmeterol concentrations. Salmeterol of high doses led to a rapid transition time (τ of about 152 sec).

Except for salmeterol at high doses, the P-DMR events induced by all agonists appeared to fit well with a one-phase exponential association process. Ligands that are known to not cause or are less effective in causing receptor internalization resulted in a rapid P-DMR event. These ligands were dopamine (t_{1/2} of about 480 seconds), catechol (t_{1/2} of about 289s), halostachine (t_{1/2} of about 349s), and salmeterol of low doses (t_{1/2} of about 370s). Ligands that are known to be effective in causing receptor internalization resulted in a slow P-DMR event with a t_{1/2} of about 520 to about 560 seconds; these ligands were isoproterenol, epinephrine, norepinephrine, and salbutamol. Interestingly, except for xamoterol, which led to a much slower P-DMR event (t_{1/2} of about 974 sec), the other partial agonists also gave rise to a slow P-DMR event (t_{1/2} of about 516 to 582 sec).

Given that the internalization directly impacts GPCR signaling, it was surmised that the differentiated kinetics in the P-DMR event may be related to internalization in part. Although not bound by theory, a receptor internalization process is believed to result in a loss in local mass density or distribution within the sensing volume of the biosensor, leading to a negative contribution to the overall response. Since dynamin is known to play an important role in the ligand-induced β₂AR internalization, we examined the effect of inhibiting dynamin activity on the ligand responses. Results showed that the pretreatment of quiescent A431 cells with 25 μM dynamin inhibitory peptide (DIPC) significantly accelerated the kinetics of the P-DMR signal induced by 2 nM epinephrine, leading to a transition time (τ) of about 120 sec and a t_{1/2} of about 200 seconds for the P-DMR event. FIG. 5 shows DMR signals of quiescent A431 cells induced by 2 nM epinephrine without (control) and with the pretreatment with 25 μM dynamin inhibitory peptide (DIPC-treated cells).

However, the pretreatment had little or no effect on the amplitude of the P-DMR event. These results suggest that receptor internalization contributes partially to both the N-DMR and P-DMR events of the epinephrine response. Conversely, the pretreatment with DIPC had little or no effect on the P-DMR signal induced by catechol or halostachine (data not shown). These results suggest that at least for catecholamine ligands the difference in kinetics of the DMR signals can be used as an indicator for their ability to cause receptor internalization.

Catechol exhibits different abilities to modulate β₂AR ligand-induced DMR signals—Catechol is a structural component of catecholamine agonists such as epinephrine and isoproterenol. The ability of catechol to modulate the DMR signals induced by other ligands was examined. FIG. 6a shows a hypothetical situation where two ligands or ligand fragments (members A and B) can bind to and recognize different binding sites and can each independently mediate different sets of cell signaling (600 or 610, respectively). In embodiments the signaling of 600 or 610 can be completely separated, or can partially overlap. FIGS. 6b and 6c show the effect of catechol co-stimulation on the β₂AR ligand-induced DMR signals. FIG. 6b shows DMR signals of A431 cells stimulated with 250 μM catechol alone (catechol) or 10 nM pindolol alone (Pindolol) were compared with cells induced by a combination of 250 μM catechol and 10 nM pindolol (Pindolol+Catechol). The simple sum of the combination of catechol and pindolol responses led to a DMR signal (Calculated) that was similar to the co-stimulated DMR signal (Pindolol+catechol). FIG. 6c shows dose-dependent responses induced by pindolol or epinephrine individually compared with responses induced by co-stimulation of either ligand with 250 μM catechol.

Results showed that the co-stimulation of A431 cells with pindolol of 10 nM and catechol of 500 μM led to a DMR signal that closely resembled, but was not identical to, the sum of the two DMR signals individually induced by pindolol of 10 nM or catechol of 500 μM (FIG. 6b). The co-stimulation led to a P-DMR event with slightly faster transition time and kinetics. Interestingly, the co-stimulated DMR signal was similar to the epinephrine response, except for the lack of its initial N-DMR event. Moreover, the co-existence of 500 μM catechol with pindolol led to an EC₅₀ almost identical to pindolol alone (FIG. 6c), and the response co-stimulated with pindolol of low doses was almost identical to the DMR induced by catechol of 500 μM alone (data not shown). Similarly, other partial agonists including alprenolol, CGP 12177 and halostachine resulted in almost identical pattern (data not shown). These results suggest that these partial agonists do not directly compete with the binding of catechol to the β₂AR, and can lead to cell signaling mostly independent of the catechol-induced signaling. However, the co-stimulation of these partial agonists with catechol still could not fully activate the receptor.

In contrast, the co-stimulation of cells with epinephrine of 100 nM and catechol of 500 μM resulted in a DMR signal that was almost identical to that induced by 100 nM epinephrine alone (data not shown). Moreover, the co-stimulation of A431 cells with epinephrine at different doses and 500 μM catechol shifted the EC₅₀ of epinephrine to the right (FIG. 6c). Similar trend was observed for strong partial agonist norepinephrine (data not shown). These results suggest that catechol competes directly with epinephrine, and both epinephrine and norepinephrine can override the catechol response at the expense of decreased potency.

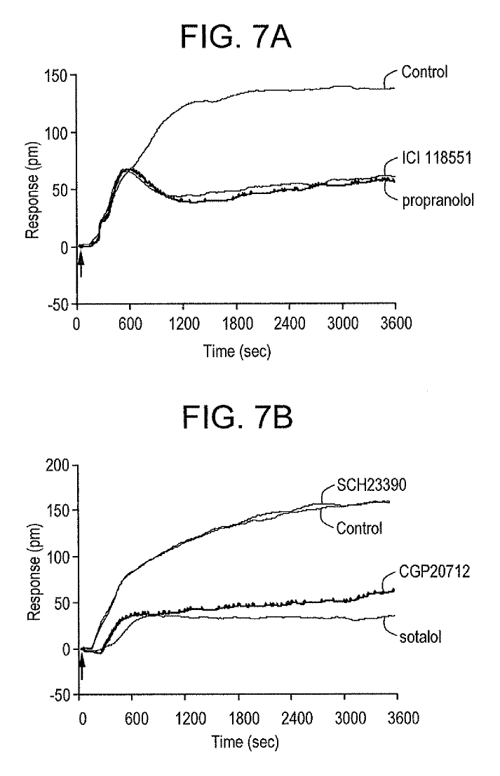

Propranolol and ICI 118551 partially attenuate the catechol-induced DMR signal—The ability of several beta blockers to modulate the catechol-induced DMR signal were examined. These beta blockers were inverse agonist ICI 118551 and propranolol, and antagonist timolol, sotalol, betaxolol, and atenolol. Neither beta blocker including ICI 118551 up to 1 μM resulted in any obvious DMR signal (data not shown). This was possibly due to relatively low constitutivity of the β₂AR in quiescent A431 cells, the low sensitivity of the biosensor assays to detect the signaling induced by inverse agonists, or both. However, all of these beta blockers dose-dependently attenuated, but were unable to completely inhibit, the catechol response (FIGS. 7a and 7b). Moreover, the catechol response in the ICI 118551- or propranolol-treated cells was different from the catechol response in the cells pretreated with other beta blockers. Both ICI 118551 and propranolol altered the dynamics of the catechol signal from a single P-DMR event to a G_{q}-like DMR signal. In contrast, the other beta blockers simply suppressed in a dose-dependent manner the amplitude and duration of the catechol signal. The apparent IC₅₀ was 1.8±1.5 nM, 193±23 nM, 46.0±11.3 nM, 415±32 nM, 5.8±1.3 nM, 0.07±0.04 mM, and 12.7±3.5 nM for propranolol, betaxolol, CGP20712, atenolol, sotalol, timolol, and ICI 118551, respectively. These results suggest that these beta blockers may not directly occupy the catechol-binding pocket, but can impact the downstream signaling induced by catechol.

FIGS. 7a and 7b show the impact of beta blockers on the catechol response: a) catechol response in quiescent A431 cells (control) compared with the response in A431 cells pretreated with 500 nM ICI 118551 or 10 nM propranolol; and b) catechol response in quiescent cells (control) compared with the response in cells pretreated with 1 μM SCH 23390, 500 nM CGP 20712, or 250 nM sotalol.

Table 1 below lists exemplary characteristics of β₂AR ligands. The binding affinity reference value (−log Kᵢ) was obtained from literature sources. The half maximal effective concentration (EC₅₀) was calculated based on the DMR response at an assay time of 50 min. The t_{1/2} was calculated based on the fitting of the P-DMR event using a single phase exponential association nonlinear regression.

TABLE 1

| | | | | | transition | |
|---|---|---|---|---|---|---|
| | −logKᵢ | | P-DMR | N-DMR | time (τ in | |
| Ligand | (ref.)¹ | −logEC₅₀ ± S.E. | (pm) | (pm) | seconds | t_{1/2} (s) |
| isoproterenol | 6.97(1) | 11.07 ± 0.07 | 232 ± 15 | 37 ± 5 | 130 ± 20 | 534 ± 32 |
| epinephrine | 7.16(2) | 10.13 ± 0.06 | 232 ± 12 | 37 ± 7 | 132 ± 20 | 521 ± 25 |
| norepinephrine | 5.40(2) | 7.99 ± 0.07 | 209 ± 16 | 29 ± 5 | 180 ± 17 | 559 ± 15 |
| dopamine | 4.35(2) | 5.96 ± 0.06 | 214 ± 32 | 31 ± 4 | 252 ± 15 | 480 ± 23 |
| catechol | 3.80(3) | 3.30 ± 0.07 | 152 ± 13 | 0 ± 3 | 130 ± 15 | 289 ± 42 |
| halostachine | 5.08(3) | 4.63 ± 0.05 | 208 ± 21 | 20 ± 7 | 200 ± 32 | 349 ± 41 |
| salmeterol | 8.89(1) | 9.68 ± 0.07 | 160 ± 14 | 0 ± 4 | 290 ± 17 | 370 ± 45 |
| salmeterol | — | 6.90 ± 0.10 | 244 ± 23 | 40 ± 11 | 152 ± 20 | n.a. |
| salbutamol | 5.35(4) | 9.07 ± 0.04 | 209 ± 13 | 32 ± 3 | 132 ± 20 | 539 ± 32 |
| labetalol | 7.97(5) | 8.05 ± 0.05 | 149 ± 17 | 8 ± 4 | 250 ± 15 | 544 ± 46 |
| cgp12177 | 9.20(6) | 9.95 ± 0.07 | 123 ± 14 | 5 ± 5 | 238 ± 26 | 582 ± 32 |
| alprenolol | 9.49(5) | 10.23 ± 0.09 | 124 ± 16 | 0 ± 4 | 233 ± 15 | 540 ± 26 |
| s(−)pindolol | 10.16(7) | 10.85 ± 0.06 | 137 ± 12 | 0 ± 3 | 249 ± 26 | 580 ± 56 |
| pindolol | 9.15(5) | 9.97 ± 0.04 | 142 ± 19 | 0 ± 4 | 250 ± 15 | 516 ± 47 |
| xamoterol | 6.06(8) | 7.32 ± 0.08 | 75 ± 18 | 0 ± 5 | 260 ± 26 | 974 ± 150 |

1. Literature references for Table 1 “−logKᵢ” comparisons.
1. Green, S. A., Spasoff, A. P., Coleman, R. A., Johnson, M., and Liggett, S. B. (1996) J. Biol. Chem. 271, 24029-24035.
2. Liapakis, G., Chan, W. C., Papadokostaki, M., and Javitch, J. A. (2004) Mol. Pharmacol. 65, 1181-1190.
3. Swaminath, G., Deupi, X., Lee, T. W., Zhu, W., Thian, F. S., Kobilka, T. S., and Kobilka, B. (2005) J. Biol. Chem. 280, 22165-22171.
4. Beer, M., Richardson, A., Poat, J., Iversen, L. L., and Stahl, S. M. (1988) Biochem. Pharmacol. 37, 1145-1151.
5. Chidiac, P., Hebert, T. E., Valiquette, M., Dennis, M., and Bouvier, M. (1994) Mol. Pharmacol. 45, 490-499.
6. Baker, J. G., Hall, I. P., and Hill, S. J. (2002) Br. J. Pharmacol. 137, 400-408.
7. Liapakis, G., Ballesteros, J. A., Papachriston, S., Chan, W. C., Chen, X., and Javitch, J. A. (2000) J. Biol. Chem. 275, 37779-37788.
8. Malta, E., Mian, M. A., and Raper, C. (1985) Br. J. Pharmacol. 85, 179-187.

TABLE 2
IC₅₀ of propranolol against different ligands.¹

| Ligand (conc.) | IC₅₀ |
|---|---|
| isoproterenol (0.1 nM) | 1.2 ± 0.3 nM |
| epinephrine (2 nM) | 2.8 ± 1.2 nM |
| norepinephrine (100 nM) | 3.5 ± 1.3 nM |
| dopamine (10 μM) | 3.6 ± 0.9 nM |
| catechol (500 μM) | 1.4 ± 0.4 nM |
| | (~50%) |
| halostachine (100 μM) | 1.9 ± 0.3 nM |
| salmeterol (100 nM) | 58.7 ± 5.7 nM |
| salbutamol (10 nM) | 2.5 ± 1.1 nM |
| labetalol (100 nM) | 9.1 ± 2.4 nM |
| CGP12177 (1 nM) | 2.1 ± 0.3 nM |
| alprenolol (10 nM) | 1.2 ± 0.5 nM |
| S(−)pindolol (1 nM) | 3.2 ± 1.9 nM |
| pindolol (1 nM) | 8.3 ± 2.3 nM |
| xamoterol (250 nM) | 3.6 ± 1.7 nM |

¹Unless specifically indicated, propranolol at high doses completely inhibited the ligand-induced responses. Propranolol at high doses only partially inhibits the catechol response (the maximum inhibition is about 50%, as shown in FIG. 7a)

### Discussion

Characteristics of β₂AR ligand-induced DMR signals—Like all GPCRs, β₂AR signaling involves a series of orderly spatial and temporal events. However, only cellular events having significant relocation of cellular material (i.e., mass) within the short sensing volume of the biosensor contribute to the overall DMR signal. The modulation profiles with panels of modulators targeting distinct downstream cascades indicated that the epinephrine response represents a collective result of several cellular events, including, for example, receptor internalization, increased cell adhesion, and cytoskeletal remodeling (data not shown).

As shown in FIG. 1, the epinephrine response consists of an initial N-DMR with a small amplitude and a short duration (about 130 sec), and a subsequent P-DMR with a large amplitude and a long duration (>10 min). Recent studies using resonant energy transfer (RET) approaches showed that in live-cells β₂AR full agonists induce detectable conformational changes within tens of milliseconds, promote rapid interaction between the receptor and G_{αβγ} complexes (t_{1/2} of about 300 msec), and lead to rapid Gₛ activation (t_{1/2}<1 sec). These rapid responses are beyond the time resolution of the current detection system. However, the slow kinetics observed here indicated that the epinephrine response is not receptor activation per se. Since it shared similarity with the forskolin-induced DMR signal and also exhibited heterologous desensitization to the forskolin-stimulated cells, the epinephrine response was mainly downstream of cAMP accumulation. The heterologous desensitization is a characteristic of cAMP-PKA signaling.

Activated Gₛ proteins activate adenylyl cyclases, thus producing cAMP with t_{1/2} of about 35 s, as measured at the whole cell level using a FRET-based sensor based on the cAMP binding domain of the exchange protein directly activated by cAMP (Epac1-camps). The activation of the β₂AR leads to rapidly increasing cAMP that is believed to be more localized initially to a particular fraction in cells due to temporally restricted diffusion. Partial agonists including norepinephrine, salbutamol, and dopamine are known to be effective in causing cAMP accumulation. All these agonists led to a DMR signal that was similar to the epinephrine response. Surprisingly, a weak partial agonist halostachine also led to a similar type of DMR signal (Table 1). The activation of β₂AR by halostachine caused a maximum increase in cAMP level that was estimated to be about 20% of that induced by epinephrine. Moreover, these ligands led to less pronounced difference in apparent efficacy, as measured based on the amplitudes of both N- and P-DMR events. However, the large observed differences in transition time and kinetics indicated that these agonists greatly differ in their ability to cause β₂AR signaling at the cell system level. Nonetheless, these results suggest that either the threshold of localized cAMP concentration resulting in the occurrence of the N-DMR event is relatively small, halostachine is effective to cause localized cAMP accumulation, or both, but not to the whole cell cAMP level. Conversely, there is no N-DMR event in the DMR signal induced by a very weak partial agonist catechol or other partial agonists that are known to be much less effective to cause cAMP accumulation. These results suggest that the biphasic DMR signals induced by full agonists or strong partial agonists are related to the increased cAMP level.

The β₂AR activation leads to rapid desensitization with a t_{1/2} of tens of seconds to several minutes. The rapid desensitization is due to the combinatorial effect of receptor phosphorylation by βAR kinase (βARK) leading to enhanced affinity for β-arrestin which blocks the re-association with G proteins, receptor phosphorylation by PKA directly resulting in uncoupling with G proteins, receptor internalization, and down-regulation. Except for the down-regulation, the other three processes occur at or near the plasma membrane and potentially involve protein trafficking, leading to changes in local mass density or distribution. Recent studies showed that the β₂AR is a member of a multivalent signaling complex organized by PKA anchoring proteins (AKAPs) including AKAP79 and Garvin. In A431 Garvin organizes the β₂AR into multivalent complexes with PKA, protein kinase C, G-protein-coupled receptor kinase 2, Src, phosphodiesterase 4, and protein phosphatase 2B; and transiently with both β-arrestin, and clathrin. The signaling complex appears to segregate into caveolae in A431, which is believed to be essential to nucleate, regulate, or propagate the β₂AR-dependent cAMP signaling. Such pre-compartmentalization may explain our current observations that there were no P-DMR event immediately following the stimulation with all β₂AR-specific ligands examined.

The β₂AR desensitization is dependent on the concentrations and types of agonists, stimulation duration, and the cellular context. Previous studies (January, B., et. al., J. Biol. Chem., 272, 23871-23879 (1997)) showed that full agonists such as epinephrine led to rapid phosphorylation with a k_{obs} of about 20 sec through both βARK- and PKA-mediated phosphorylation. In comparison, partial agonists such as ephedrine resulted in a delayed and reduced phosphorylation, mainly by PKA. Whereas the recruitment of β-arrestin to the βARK-phosphorylated β₂AR is relatively slow with a t_{1/2} of about 2.5 min. As illustrated in FIG. 3a, the transition time for the initial DMR phase to the P-DMR phase was dose-dependent for a given ligand; the higher the ligand concentration the shorter the transition time. Furthermore, the transition time was also ligand-dependent. At a saturating concentration, full agonists such as epinephrine generally led to faster transition time than partial agonists such as dopamine (Table 1). Since the observed transition time appears to correlate well with the kinetics of receptor desensitization, we surmised that the receptor desensitization may be a rate-limited step in controlling the transition from the initial DMR to the subsequent P-DMR event.

The present biosensor assays, if desired, can continuously monitor a ligand-induced DMR signal during a long period of time after the agonist addition. It was demonstrated that epinephrine led to a rapid increase in receptor phosphorylation, followed by a slow decay. As a result, after prolonged persistent stimulation (about 30 min) the epinephrine-induced phosphorylation is close to those induced by the weak partial agonist ephedrine and dobutamine. As shown in FIG. 3, the potencies obtained for both epinephrine and norepinephrine were found to be a function of the assay time. The longer the assay time the higher and bigger the shift relative to binding affinity the potency. These results are surprising, considering that the potency of epinephrine to stimulate receptor phosphorylation by GRKs (about hundreds of nM) is about several orders of magnitude higher than for PKA site phosphorylation (about nM range). One possibility is that a portion of βARK-mediated phosphorylation not only increases in proportion to the coupling efficiencies of agonists, but also increases over time particularly for partial agonists, leading to signaling that may act synergistically with the PKA-mediated signaling to cause the P-DMR. In this context, it is reasonable that the potency of epinephrine, due to its higher coupling efficiency, would exhibit much more significant dependency on assay time than norepinephrine; and partial agonists that are known to be able to cause cAMP accumulation may be effective to provide DMR signals comparable to the full agonist epinephrine. These predications were consistent with our observations (see e.g., FIG. 1, FIG. 3, and Table 1). The shift in apparent EC₅₀ relative to affinity was found to be ligand-specific (FIG. 4). Although not bound by theory, the differential shift observed may be indicative of the ability of ligands to cause βARK phosphorylation and sequential signaling. The ligands resulting in a larger shift may have higher coupling efficiency, leading to effective cAMP accumulation and subsequent receptor phosphorylation by both βARK and PKA. However, the ligands providing smaller shifts may not be effective to promote cAMP production, but effective to promote PARK phosphorylation and sequential signaling.

The β₂AR internalization lags slightly behind desensitization through both clathrin- and caveolae-dependent mechanisms in A431 cells. The receptor internalization would lead to a decreased local mass density. However, in the epinephrine DMR signal, a prolonged P-DMR phase followed a small initial N-DMR, suggesting that the receptor internalization may not contribute to the overall DMR signal, or alternatively, is not detectable by the biosensor. To clarify this, the effect of dynamin inhibitor on the epinephrine response was examined, since the β₂AR is known to internalize in a dynamin-dependent manner. Results showed that the inhibition of dynamin activity greatly accelerated the kinetics of the P-DMR event, without any significant effect on the total amplitudes of the P- and N-DMR (FIG. 5). Although not limited by theory, an explanation may be that the negative contribution of receptor internalization to the overall DMR signal indeed takes place during the P-DMR phase, and is overwhelmed by a positive DMR event (possibly due to increased degree in cell adhesion; data not shown). The kinetic analysis of the P-DMR event supported such hypothesis. The t_{1/2} was estimated to be around 530 sec for the P-DMR induced by the full agonists epinephrine and isoproterenol. The strong partial agonists including norepinephrine and salbutamol that are known to promote internalization effectively also led to similar t_{1/2}. In contrast, the weak partial agonists such as dopamine, catechol, and halostachine that are not effective to cause receptor internalization resulted in a smaller t_{1/2} (i.e., faster kinetics). These results suggest that the kinetics of the P-DMR event can be used as an indirect measure of the ability of ligands to induce receptor internalization.

The β₂AR activation stimulates MAPK signaling, through G protein-dependent and independent pathways. In addition, the β₂AR activation ultimately leads to cytoskeletal remodeling and an increase in cell adhesion (data not shown). Ligand structure and functional selectivity—Epinephrine, isoproterenol, norepinephrine, and dopamine are structurally similar catecholamine ligands. These four ligands led to a similar type of DMR signal, which consisted of a small N-DMR with a short duration and a large and prolonged P-DMR (FIG. 1). All four ligands are known to be able to produce comparable cAMP through the β₂AR, but differ greatly in causing receptor internalization. Although not limited by theory, dopamine is believed to be a minimal structure required for the activation of both a rotamer toggle switch in the TM6 and an ionic-lock between the TM3 and TM6. In contrast, a catecholamine structural component, such as catechol, can activate the rotamer toggle switch but not the ionic-lock, and can act as a very weak partial agonist to activate Gₛ, leading to a very small increase in cAMP but not receptor internalization. Consistent with these findings was that catechol was not able to produce an epinephrine-like signal whose DMR consists of a initial steady phase with short duration and a sequential P-DMR with a smaller amplitude. Furthermore, besides the different amplitudes of both P- and N-DMR events, these ligands resulted in different kinetics including transition time and t_{1/2} of the P-DMR, which seems to correlate well with their ability to activate Gₛ and stimulate receptor internalization. Interestingly, halostachine, a weak partial agonist that only consists of the β-hydroxyl and N—CH₃, was also able to provide an epinephrine-like DMR signal.

Salbutamol, salmeterol, and labetalol are three structurally related non-catechol ligands, and have a non-hydroxyl group in the meta-position of the aromatic ring (see FIG. 1). These three ligands exhibited significant differences in mediating DMR signals. Salbutamol provided a DMR signal that was similar to the epinephrine response. Labetalol provided a DMR signal that was between the catechol and epinephrine responses. The DMR signal induced by labetalol also consisted of a noticeable N-DMR and a prolonged P-DMR, but with much smaller amplitudes, compared to the salbutamol or epinephrine response. Salmeterol resulted in a biphasic dose-dependent response having two well-separated transition times (290 s and 152 s) and EC₅₀ values (0.12 nM and 130 nM). At low doses, the DMR signal induced by salmeterol was similar to the labetalol response, while salmeterol at high doses (>100 nM) led to a salbutamol-like DMR signal. Salmeterol is a long acting β-agonist with low intrinsic activity and a very slow rate of onset of action, characteristics determined in part by the very hydrophobic nature of the compound. Salmeterol binds to the active site (Ser-204, Ser-207 and Asp-113) of the β₂AR and also binds to its exosite, and results in a slow internalization of the β₂AR. Furthermore, salmeterol exhibited dual efficacies: a very weak partial agonist for producing an effective interaction between the receptor and β-arrestin 2, and a full agonist of cAMP accumulation in C2C12 cells stably expressing the β₂AR. In HEK293 cells, despite stimulating GRK-mediated receptor phosphorylation after 30 minutes to an extent similar to those observed with agonists of high intrinsic efficacy such as epinephrine, salmeterol at 50 nM did not induce significant β₂AR internalization and was incapable of stimulating the translocation of enhanced green fluorescent protein-arrestin 2 chimera to the cell surface.

The four structurally related beta-blockers alprenolol, CGP12177, S(−) pindolol, and pindolol all provided a catechol-like DMR signal, but with distinct kinetics. CGP 12177 is a known partial agonist of the β₂AR, and is capable of stimulating Gₛ. The co-stimulation of cells with pindolol and catechol (500 μM) led to a DMR signal that was close to the sum of the two DMR signals obtained individually. Furthermore, the presence of catechol did not alter the EC₅₀ of pindolol. Similar signals were also observed for the other three ligands. In contrast, the co-existence of catechol shifted the EC₅₀ of epinephrine or norepinephrine to the right. These results suggest that these structurally related beta blockers do not compete directly with- or indirectly influence the binding of catechol, and mediate signaling mainly independent of catechol.

Other structurally diverse beta-blockers, including inverse agonist propranolol and ICI 118551, provided little or no DMR signals. However, these beta blockers dose-dependently attenuated the catechol response, with an IC₅₀ close to their corresponding binding affinity and a maximum inhibition of about 50-60%. The catechol response in the propranolol- and ICI 118551-pretreated cells was considerably different from the cells pretreated with other beta blockers (FIG. 7). These results were surprising, considering that a recent in vitro biophysical study showed that β₂AR inverse agonist ICI 118551 had little or no effect on the catechol-induced fluorescence changes of a fluorescently engineered β₂AR. It was believed that ICI 118551 does not occupy the catechol-binding pocket, and is not able to inhibit β₂AR activation by catechol. In these studies, the differences in conformational changes were analyzed based on the changes between only two points within an engineered receptor, and their functional consequences were largely limited to single signaling pathways or cellular events. In contrast, the biosensor-based methods of the present disclosure monitor an integrated DMR response in real time, which closely represents ligand-induced cell signaling at the cell system level. The results obtained in the present disclosure suggest that these beta-blockers might not directly compete with catechol, but may instead lead to a conformational change that directly impacts the catechol signaling, or alternatively, results in structural change(s) of the receptor that may in turn have direct functional consequence(s).

The disclosure has been described with reference to various specific embodiments and techniques. However, it should be understood that many variations and modifications are possible while remaining within the spirit and scope of the disclosure.

### REFERENCES

1. Fang, Y., et al., Biophys. J, 91, 1925-1940 (2006).
2. Fang, Y., et al., J. Pharmacol. Toxicol. Methods, 55, 314-322 (2007).
3. Fang, Y., Assays and Drug development Technologies. 4: 583-595 (2006).
4. Simmons, M. A., “Functional selectivity, ligand-directed trafficking, conformation-specific agonism: what's in a name?”, Mol. Interv., 5, 154-157 (2005).
5. Urban, J. D., et al., “Functional selectivity and classical concepts of quantitative pharmacology,” J. Pharmacol. Exp. Ther., 320, 1-13 (2007).
6. Fang, Y., et al., “Label-Free Biosensors and Cells,” PCT App. No. PCT/US2006/013539 (Pub. No. WO 2006/108183)(Attorney Docket No. SPO5-037).

What is claimed is:

1. A method for determining ligand-directed functional selectivity of a receptor of a live-cell, the method comprising:
immobilizing a live-cell having a selected receptor on a biosensor;
contacting the immobilized live-cell with a molecular fragment of a ligand of the receptor;
contacting the molecular fragment contacted immobilized live-cell with a stimulus; and
detecting and comparing the stimulus-induced biosensor response of the live-cell in the presence and absence of the molecular fragment.

2. The method of claim 1 wherein contacting with a molecular fragment and contacting with the stimulus compound is accomplished simultaneously, sequentially, or in reverse order.

3. The method of claim 1 wherein the molecular fragment comprises at least one structural component of a natural ligand of the receptor.

4. The method of claim 1 wherein the molecular fragment is a structural component of a natural agonist for the receptor.

5. The method of claim 4 wherein the receptor is selected from a β₁-adrenergic receptor, a β₂-adrenergic receptor, and a combination thereof, and the molecular fragment is selected from catechol, halostachine, or a combination thereof.

6. The method of claim 1 wherein the molecular fragment comprises a partial sequence of a natural ligand if the receptor ligand is a peptide or protein

7. The method of claim 1 wherein the receptor comprises a G protein-coupled receptor (GPCR).

8. The method of claim 7 wherein the G protein-coupled receptor (GPCR) comprises a β₂-adrenergic receptor (β₂AR).

9. The method of claim 1 wherein the biosensor response comprises the dynamics, the phases, the signal amplitude, the kinetics of each phase, the transition time from one phase to another phase, or a combination thereof.

10. The method of claim 1 wherein the biosensor response comprises a dynamic mass redistribution if an optical biosensor is selected, a bioimpedance signal if an electrical biosensor is selected, or a combination thereof.

11. The method of claim 1 wherein the stimulus comprises at least one of a natural agonist, a full agonist, strong partial agonist, an antagonist, an inverse agonist, a partial agonist, or a combination thereof.

12. The method of claim 1 wherein the biosensor comprises a label-free apparatus that accomplishes a label-free detection method.

13. The method of claim 1 wherein the molecular fragment modulates cell signaling and the stimulus agonizes, antagonizes, or is independent of the cell signaling response.

14. A method for molecular fragment-based functional screening against a receptor of a live-cell using label-free biosensor, the method comprising:
immobilizing a live-cell having a selected receptor on a biosensor;
contacting the immobilized live-cell with a first molecular fragment;
contacting the immobilized live-cell with a second molecular fragment; and
detecting and comparing the molecular fragment-induced biosensor response of the live-cells.

15. The method of claim 14 wherein contacting with the first molecular fragment and contacting with the second molecular fragment is accomplished simultaneously, sequentially, or in reverse order.
